(19) United States
(12) Patent Application Publication
(10) Pub. No.: US 2010/0255088 A1
(43) Pub. Date: Oct. 7, 2010

(54) Method for delivering a combination of resveratrol and aspirin for use in treatment and prevention of vascular disease

(76) Inventor: Yoel Ovil, Johannesburg (ZA)

Correspondence Address: Dara L. Onofrio, Esq.;c/o ONOFRIO LAW, Suite 225, 15 N. Mill Street, Nyack, NY, 10960, US

(21) Appl. No.: 12/798,466

(22) Filed: Apr. 5, 2010

Related U.S. Application Data
(60) Provisional application No. 61/212,006, filed on Apr. 6, 2009.

(51) Int. Cl. A61K 9/48; A61K 31/616; A61P 9/10; A61P 43/00

(52) U.S. Cl. 424/463; 424/451; 514/165

(57) ABSTRACT

A method for delivering a composition for use in treatment and prevention of vascular disease by providing a vehicle for delivery of the composition to an individual, wherein the composition contains a combination of aspirin and resveratrol; administering the vehicle to the mouth of the individual, wherein the vehicle melts at the body temperature of the individual such that the aspirin and resveratrol is absorbed thru the oral mucosa and delivered directly into the inidvidual's bloodstream; and a related melting capsule comprised of a material that has the same melting point as the human body temperature and includes aspirin in the range of 81 to 325 mg and resveratrol in the range of 10 to 300 mg.

This application claims the benefit of U.S. provisional application no. 61/212,006 filed Apr. 6, 2009, which is incorporated herein in its entirety by reference.

### FIELD OF THE INVENTION

The present invention relates to a method for treatment and prevention of coronary, cerebral and peripheral vascular disease using a combination of reseveratrol and aspirin. In particular, the method provides delivery of a combination of resveratrol and aspirin via oral mucosal absorption for the treatment and prevention of vascular disease.

### BACKGROUND OF THE INVENTION

Aspirin taken everyday either low or high doses is known to reduce coronary events any where from 30% to 50%. Aspirin is a strong cox1 inhibitor, the enzyme needed for the conversion of arachnoidic acid to the mother compound of prosyaglandins PGH2. As a consequence three main prostaglandins production are decreased:
1. The reduced thrombaxane production will lead to marked reduction of platelet aggregation and prevention of clot formation this is how aspirin is prolonging life.
2. The reduced prostacycline production will lead to reduced endothelial function (Inner layer of cell inside the blood vessel)causing rough surface with devastating results as far as clot formation is concerned. The net effect of aspirin reducing clot formation is just because the thrombaxane effect prevails.
3. The reduction of PGE2 is associated with marked reduction of stomach lubrication leading to fatal GI complications caused by aspirin treatment.

It is therefore clear that the aspirin treatment is associated with two major drawbacks: severe GI complications and devastating damage to the blood vessels.

Further more recent studies have proven that the prevention of vascular disease should be directed toward not only to platelet aggregation and endothelial cells but also reducing high cholesterol levels in the blood as well as enhancing vasodilatation and prevention of muscular vessels hypertrophy. None of these important goals are addressed by aspirin treatment alone.

Resveratrol the molecule found in the red wine is also a cox1 inhibitor that acts in a completely different way. The aspirin via the acetyl group is inflicting irrevocable damage to the enzyme inside the platelets that is permanent and lasting two weeks the life time of a platelet whereas the resveratrol is inhibiting the gene responsible for the production of cox1. The action of resveratrol is dose dependent and unfortunately blood levels are not sustainable due to rapid kidney excretion and liver degradation. Higher blood levels could be reached through sublingual absorption but definitely not sustainable,

Compared to aspirin, resveratrol has a vast and positive effect on the secretion of prostacycline, increasing it, notably leading to marked improvement of endothelial function. Further more resveratrol acts as very potent antioxidant, reducing LDL (bad cholesterol) increasing HDL (good cholesterol). It also acts as a very potent anti platelet aggregation through thromaxane reduction even in a group of patients termed “aspirin resistant.” Despite good levels of aspirin in the blood thrombaxane levels did not come down. Resveratrol was also found to induce marked vasodilatation through the NO system as was proven in isolated retinal artery vessel in pigs. Resveratrol ability to prevent muscular vessel hypertrophy in combination of aspirin is believed to achieve optimal prevention of vascular disease.

The finding that resveratrol can act as a proton pump inhibitor reducing HCL secretion by the parietal cells in the stomach thus counter acting the aspirin action of reduced lubrication due to inhibition of PGE2 synthesis further supports the cardiovascular benefits of combining of resveratrol and aspirin

In acute myocardial infarction and unstable angina aspirin has to be delivered to the blood stream in the fastest way in order to prevent further damage to the myocardium. The aspirin is not water-soluble and cannot be administrated intravenously. Oral Administration the stomach will take 20 or 30 minutes such a long period can inflict undesired and irrevocable damage to the ischemic myocardium. Chewable aspirin can reach the blood stream relatively fast seven to ten minutes however most of these patients are either non cooperative or unconscious and such a method is not applicable. Further more recent studies have shown that GI complications associated with aspirin treatment are originated from stomach irritation of the acetyl salicylic acid itself.

Resveratrol administrated sublingually was found to have far better bioavailability than resveratrol administrated orally. One mg of resveratrol administrated sublingually has reached blood level of 37/nan/ml in few minutes after the sublingual administration this blood level is equivalent to oral administration of 250 mg of resveratrol. Sublingual administration of the proposed combination of drugs appears to offer undoubted advantages, including:
1. Very fast delivery system of the said medications to the blood stream.
2. Reduction of GI complication associated with oral administration of aspirin due to bypassing of the stomach.
3. Improved bioavailability of the resveratrol; higher blood levels of resveratrol; as well as more sustainable levels due to reduced first pass reaction in the liver(absorbed medication through the stomach gets to the liver to be inactivated there).

A comprehensive background and understanding of the pathogenesis of atherosclerosis disease as well as vast knowledge of the medications mode of action is needed in order to find the right combination of drugs to achieve the ultimate goal to stop this inflammatory process which has become the number one killer in the western world.

Four elements are targeted:
1. platelets;
2. endothelial cells;
3. hyperlipidemia; and
4. vessel wall muscle.

The present invention provides a method for treatment and prevention of vascular disease by providing a vehicle for delivery to an individual containing a combination of aspirin (acetyl salicylic acid) and resveratrol; and administering the vehicle to the mouth of the individual, wherein said vehicle melts at the body temperature of the individual such that the combination of aspirin and resveratrol is absorbed thru the oral mucosa and delivered directly into the bloodstream. The drugs works synergistically together and also correct the main drawbacks of administering each drug separately and by ingestion, leading to a more efficient and safer drug.

The vehicle itself is preferably a medicinal melting capsule, but may also be a solution of the combination of aspirin and resveratrol; a tablet of the combination of aspirin and resveratrol; or a powder including the combination of aspirin and resveratrol. In all embodiments, all vehicles are administered orally for absorption thru the oral mucosa, preferably sublingual, and direct delivery into the bloodstream.

Sublingual absorption carries ample of advantages such as very fast blood level rise, no food, enzymes and first pass liver deactivation allowing smaller and accurate dosages and no gastrointestinal complications. The main obstacle remains the buccal mucosa that can absorb only through the passive absorption only certain chemical compounds can be absorbed easily and fast like the resveratrol other compounds due to their size chemical structure and solubility are hardly absorbed such as the aspirin. Such compounds have to go through a certain modifications that on the one hand will maintain their original active ingredient to keep their chemical activity but on the other hand will allow easy and swift absorption.

Previous attempts to achieve sublingual absorption of aspirin and caffeine molecule ergotamine had failed with absorption rate of less than 5%. The inventors believe the absorption of ergotamine across the buccal mucosa appears to be passive process, pH dependent but independent of ergotamine concentration or the simultaneous presence of caffeine. Because of the low solubility of the pH of the saliva it is unlikely that therapeutically useful amounts of the drug would have absorbed across the buccal mucosa even after the drug had been in the mouth for five minutes, Journal of Neurology, Neurosurgery & Psychiatry 1974, October 37 (10).

In order to achieve optimal buccal absorption it is not enough to have the active ingredient adjusted or modified for the passive absorption but a proper delivery system has to be developed to ascertain complete isolation of the active ingredient from oxygen heat light and the capsule wall on the one hand but to allow instant and complete release of the material when usage is needed. The medicinal melting capsule was developed and chocolate was found to be the optimal material to achieve the said goals. It can maintain the active ingredient powder or liquid (great advantage) in perfect stable condition until it is used under the tongue. The chocolate is so designed to form with the active ingredient (powder or liquid) a thick and sticky mixture (binders will be used in the chocolate manufacturing) that will adhere to the oral mucosa to ensure maximal absorption time as needed and to prevent spillage or aspiration in cases of unconscious patients. The chemical composition of the chocolate will also be subjected to chemical changes required for optimal absorption. If basic or acidic environments are needed, sodium hydroxide or bicarbonate will be added accordingly to meet the optimal conditions for perfect absorption.

The quantity of the chocolate capsule will vary according to the nature of the active ingredient. If the active ingredient is liquid more chocolate is needed in order to prevent any leak prior the actual use and to achieve the optimal thick sticky mixture to ensure proper absorption with no spillage or aspiration.

Further more recently it has been discovered that the chocolate contains strong anti oxidant properties lowering the LDL (bad cholesterol) increasing the HDL (good cholesterol) as well strong anti platelet aggregation qualities. Any type of chocolate may be used. It is essential however that in certain applications, it will contain no sugar. Manitol can be used and should be used to sweeten the pill.

The medicinal melting capsule is designed to provide a solution to overcome these problems by melting instantly in the mouth of the individual allowing the inside content to be absorbed directly through the mucosa immediately into the bloodstream without any gastric distress or degradation of the medication.

The prior art has shown various oral and buccal delivery systems for therapeutic agents and active ingredients. See U.S. Pat. Nos. 7,018,653; 6,488,953; 6,183,775; 5,827,525; 5,035,252 and U.S. Patent Publication No. 2008/0317850 but none disclose the medicinal melting capsule as described herein.

The present invention method is designed to achieve the following goals.

1. To combine aspirin and resvertrol in order to achieve, optimal anti platelet aggregation, to preserve ideal endothelial function, to enhance arterial vasodilatation, to prevent vessel wall muscular hypertrophy, to act as a strong anti oxidant as well as reducing the LDL levels and increasing the HDL levels to prevent plaque formation inside the blood vessels thus preventing coronary, cerebral and peripheral vascular disease.

2. To combine aspirin and resveratrol to achieve an ideal anti-inflammatory combination to prevent malignancies, each and one of them is acting in a different way in an aim to reach perfect synergism.

3. To combine aspirin and resveratrol in order to minimize the gastric damage caused by the aspirin. Resveratrol acting as proton pump inhibitor working on H+/K+ ATPase can reduce dramatically HCL production by parietal cells thus compensating for the inadequate gastric lubrication caused by the aspirin.

4. To combine aspirin and resveratrol in order to achieve optimal Cox1 inhibition, where the aspirin is causing long standing (two weeks) irrevocable damage to the Cox1 enzyme securing long and very effective platelet aggregation inhibition. The resveratrol instead is inhibiting the gene responsible on the synthesis of Cox1 it's action is short term and is dependent on optimal blood levels of resveratrol which are very difficult to achieve due to the very fast elimination of resveratrol and it's metabolites from the blood.

5. To combine aspirin and resveratrol in cases of proven bacterial, fungal, or viral infection where the resveratrol is capable to destroy the external pathogen (bacteria, virus, fungus) and the aspirin will supply anti-inflammatory cover to reduce pain, fever and other signs of acute inflammation.

6. To combine aspirin and resveratrol in order to treat type 2 diabetes where both drugs are synergistically working as anti platelet aggregation to prevent further damage to the coronary, cerebral, and peripheral vessels, and the resvertol will act as an insulin like drug reducing the blood sugar levels(in both forms low insulin and insulin resistance) and normalizing the associated hyperlipidemia.

7. To combine aspirin and resveratrol in such a way that the aspirin is in form of aqueous buffered solution with a pH ranging from 2 to 4 to enable instant and very efficient sublingual absorption of the aspirin allowing immediate blood levels rise of aspirin which is so critical in acute myocardial infarction, as well as complete bypassing of the stomach reducing the GI complications associated with aspirin treatment

8. To combine the aspirin and resveratrol in any doses needed but preferably full dose of aspirin (full dose is needed for cancer prevention, and achievable due to resveratrol stomach protection) and high dose of resveratrol (500 mg) due to very short half life time in the blood as well as almost no adverse effects in rats and human.

9. To combine the aspirin and resveratrol in order to achieve far better anti-inflammatory reaction due to the synergistic effect of these two drugs in severe arthritis, this combination of drugs can allow far higher aspirin dosages. Gastric mucosa protection is achieved by the resveratrol (proton pump inhibition action) and complete bypassing of the stomach achieved the sublingual absorption.

10. To combine aspirin and resveratrol to prolong life. Each one of the two components is capable in reducing mortality due to coronary, cerebral, peripheral vascular disease and malignancy in it's own unique way.

Resveratrol is capable of prolonging life by shifting the main metabolism from high caloric metabolism to low caloric metabolism regardless of the amount of calories supplied to the plant or the animals. By activating two enzymes sir1 and sir2 resveratrol shifts the metabolism from high calories to low calories (regardless the amount of calories fed) thus doubling the life span of yeast and primitives animals that do not die from either vascular disease or from malignancies. These superb results of resveratrol were reached in tissue cultures where high blood levels of resveratrol were artificially maintained. Whether these levels can be reached and sustained in order to be able to replicate it in humans is the subject of the invention process.

The aspirin, at least 325 mg daily taken for ten years, is known to reduce mortality in 50% of the patients. Higher doses taken for ten years reduced mortality in 75% of the patients. Though aspirin mode of action is completely different from resveratrol chemical action as explained above and has got nothing to do with metabolic shift the fact that it can produce such excellent results and that the main risk gastrointestinal complications is almost abolished with the invention method delivery of aspirin and resveratrol in a sublingual absorption where the resveratrol acts as proton pump inhibitor and the sublingual absorption of the aspirin prevents local injury to the gastric mucosa)is another excellent reason for the combination of these two outstanding anti inflammatory drugs.

### SUMMARY OF THE INVENTION

In the present invention, these purposes, as well as others which will be apparent, are achieved by providing a method for delivering a composition for use in the treatment and prevention of vascular disease comprising the steps of providing a vehicle for delivery of the composition to an individual; wherein the composition contains a combination of aspirin and resveratrol. Administering the vehicle to the mouth of the individual, wherein the vehicle melts at the body temperature of the individual such that the combination of aspirin and resveratrol is absorbed thru the oral mucosa and delivered directly into the bloodstream.

Preferably the delivery vehicle is a medicinal melting capsule comprised of a material that has the same melting point as the human body temperature. This medicinal melting capsule is more fully described in the simultaneously filed patent application entitled: MEDICINAL MELTING CAPSULES FOR ORAL MUCOSAL ABSORPTION, which is incorporated in its entirety herein by reference.

The vehicle may also be a solution of the combination of aspirin and resveratrol; a tablet of the combination of aspirin and resveratrol; or a powder including the combination of aspirin and resveratrol. In all embodiments, all vehicles are administered orally for absorption thru the oral mucosa, preferably sublingual, and direct delivery into the bloodstream. The vehicle includes aspirin in the range of 81 to 325 mg; and resveratrol in the range of 10 mg to 300 mg.

The invention also specifically provides a melting capsule comprised of a material that has the same melting point as the human body temperature and includes aspirin in the range of 81 to 325 mg and resveratrol in the range of 10 to 300 mg.

The invention delivery of the drugs thru the oral mucosa allows fast delivery to the bloodstream. It reduces aspirin related gastrointestinal problems and higher more sustainable levels of resveratrol, compared to oral ingestion.

Other objects, features and advantages of the present invention will be apparent when the detailed description of the preferred embodiments of the invention is considered which should be construed in an illustrative and not limiting sense.

### DETAILED DESCRIPTION OF THE INVENTION

The term aspirin is used interchangeably with its chemical name acetyl salicylic acid. The two terms are meant to mean the same thing.

In accordance with the present invention a method for treatment and prevention of vascular disease is provided by simultaneously delivering a therapeutic amount of aspirin and a therapeutic amount of resveratrol to a patient.

Aspirin provides outstanding anti platelet aggregation activity but it was recently discovered that a significant group of patients despite being treated with aspirin developed an acute coronary event. Further investigation of this group of patients has revealed a resistance to aspirin and an inability of the aspirin to reduce platelet aggregation despite adequate drug levels in the blood. This resistance group was alternately treated with resveratrol which resulted in optimal platelet aggregation inhibition. This is a clinical evidence that combined treatment of aspirin and resveratrol is superior and can prevent coronary events from major group of patient.

Serious gastrointestinal complications remain the main threat and fear of the majority of the population to use aspirin as a preventive measure despite clinical results demonstrating the benefit to taking “an aspirin a day”. Attempts to coat the aspirin or to mix it with a buffer solution had failed to achieve significant reduction of these deadly complications.

It is generally known that the group of drugs called proton pump inhibitors reduce dramatically the hydrochloric acid (“HCl”) production by the parietal cells in the stomach thus significantly reducing the gastrointestinal complications associated with long usage of aspirin. This group of drugs works on the H+/K+ ATPase pump which reduces the release of H+ thus preventing the production of hydrochloric acid almost abolishing the rate of gastrointestinal complications. The results were so good that medically and economically it was advocated to combine use of aspirin and a proton pump inhibitor in order to achieve the ultimate outcome to eliminate gastrointestinal complications. However, it was recently discovered that long term treatment with proton pump inhibitors (“ppi”) is associated with high rate of femoral fractures, which convinced the medical community not to use the said combination as prophylaxis regime since the risks outweighed any benefits.

It has been reported that resveratrol, also being a Cox1 inhibitor, does not damage the gastric mucosa as aspirin does but on the contrary it protects the gastric mucosa working on the H+/K+ ATPase, the same way the proton pump inhibitors did, thus reducing significantly the hydrochloric acid production (Life Sciences, Volume 66, Issue 8, 14 Jan. 2000 pages 663-673).

The low hydrochloric acid production induced by the resveratrol is believed to compensate for the inadequate lubrication induced by the aspirin therapy thus has the synergy between resveratrol and aspirin is very beneficial and life saving.

The pharmacokinetics bioavailability and mode of action of resveratrol and aspirin are completely different. Aspirin through acetylation of the enzyme carboxyloxigenaze inside the platelet irrevocably destroys the enzyme thrombaxane synthetase thus preventing it from producing thrombaxane thereby less aggregation of the platelets is achieved. The permanent destruction of the enzyme lasts two weeks which is the life time of the platelet. This mode of action can assure low blood levels of thrombaxane far and beyond the levels achieved by the resveratrol.

The resveratrol in comparison to the aspirin inhibits the synthesis of carboxyloxygenase through inhibition of the gene responsible for its synthesis there is no irrevocable damage to the enzyme and the duration of action is uncertain. Further more the bioavailability of resveratrol is relatively low due to it's rapid metabolism and elimination. Most of resveatrol administrated through the mouth is conjugated and metabolized and reaches peak plasma levels sixty minutes after administration. It is still unknown how active the conjugated resveratrol is. It is therefore the goad of the present invention that simultaneous administration of a combination of resveratrol and aspirin provides longer duration and safer action on thrombaxane level reduction.

Better understanding of the pathogenesis of atherosclerosis heart disease has brought us to the point where we understand the four elements associated with the plaque formation which is the cause of vascular disease. Aspirin has been proven to be a very effective tool as far as prevention is concerned but works on only one element of the four. Resveratrol works simultaneously on all four elements leading to far and better prevention than use of aspirin treatment only.

Beside platelets aggregation inhibition through reduction of thrombaxane levels which is executed by aspirin and resveratrol in different ways the function and integrity of the endothelial cells is as important as anti platelets aggregation or even more. Prostacycline is a special prostaglandin secreted by the endothelial cell themselves. High secretion of prostacycline indicates good function of the endothelial cells and low secretion reflects sick and rough endothelium, which will trigger platelet aggregation.

Resveratrol increases prostacycline in the blood vessels improving endothelial function and decreasing notably the stimulus for platelet aggregation. The aspirin is decreasing prostacycline secretion, thus reducing endothelial function.

Hyperlipidemia in particular high LDL and low HDL will increase the risk of plaque formation, therefore it is imperative to lower the LDL as much as possible and to increase the HDL to protective levels. The resveratrol provides this function and in addition is a very potent antioxidant, which prevents the LDL to be oxidized.

Aspirin has no known effect on either HDL nor on LDL levels.

The last element in enhancing arterial plaque is the vessel wall smooth muscle. It has been proven on isolated pig retinal artery that resveratrol has strong vasodilating effect through the mediation of the NO system and prostacycline secretion. The ability of the vessel to dilate and in so doing to absorb all the enormous shearing forces involved was proven to be essential to prevent plaque formation. In addition resveratrol was found to inhibit wall muscle hypertrophy, which was found to be as an important enhancing factor in plaque formation. Aspirin has no known effect on wall muscle hypertrophy or vasodilatation

The aspirin by inducing irrevocable damage to Cox1 can induce very potent and long lasting anti platelet aggregation action that has proven itself clinically without a doubt, however being a non-selective Cox1 inhibitor it causes severe gastrointestinal complications. Furthermore the aspirin despite being the best drug for anti platelet aggregation does not have any effect on hyperlipidemia endothelial cell function and integrity and the muscle of the vessel wall.

The resveratrol has less aggressive Cox1 inhibition that has shorter duration however it is still excellent Cox1 inhibitor that can reduce the levels of thrombaxane even in aspirin resistant patients. It protects the stomach like the proton pump inhibitors from the aspirin damage. In addition it acts as an excellent antioxidant increasing HDL and decreasing LDL, and protecting the endothelial cells as well as the vessel wall.

Both aspirin and resveratrol were found to play a major role in cancer prevention. Resveratrol has been found to inhibit the proliferation of a variety of human cancers induced by chemical carcinogens. The incidence of various human carcinomas in patients treated with full dose aspirin for ten years and above was halved. It appears that the inflammatory reaction plays a major role in the pathogenesis of various malignancies.

Resveratrol was found to have other than antiviral, antifungal, and antibacterial activities as well as major metabolic impacts. It has insulin like effect in type2 diabetes lowering the sugar levels and normalizing the hyperlipidemia, and it also activates two major enzymes associated with low calorie diets, specifically sir1 and sir2. By so doing it was managed to double life span of primitive animals and plants.

Resveratrol is not known to be toxic or cause adverse effects in humans, but there have been only a few clinical trials to date. A recent trial that evaluated the safety of oral resveratrol in ten subjects found a single dose up to 5 grams resulted in no serious adverse effect. In rats, daily oral administration of trans resveratrol at doses up to 300 mg/kg of body weight for four weeks resulted in no apparent adverse effects.

The clinical data will support the synergistic effects between these two outstanding anti-inflammatory drugs. The simultaneous usage of these anti-inflammatory drugs provides far better prevention of vascular disease and is free of major adverse effects as well as providing better understanding of cancer prevention via the anti-inflammatory drugs.

Despite such high doses of resveratrol administrated to human and rats without any side effects there are few clinical conditions based on our knowledge today that should be regarded as absolute contraindication for resveratrol treatment.

The chemical structure of resveratrol is very similar to the synthetic estrogen therefore it can easily bind to the estrogenic receptors, but in different environments it reacts differently, an estrogen agonist under some conditions and estrogen antagonist under other conditions. Therefore it is advisable that in estrogen receptor positive breast cancer not to treat the patient with resveratrol. Resveratrol may inhibit children growth thus it should be avoided from children and breast-feeding mothers. Resveratrol is capable of inhibiting the expression and activity of certain cytochrome p450 enzyme in the liver leading to marked reduction or enhancement of break down of other drugs taken by the patient this may bring upon undesired levels of the medications in the blood including intoxications.

Currently the main concern remains the bioavailability i.e. to reach optimal and sustainable blood levels of resveratrol and it's metabolites to achieve the known goals. Although transresveratrol appears to be well absorbed by humans when taken orally, its bioavailability is relatively low due to it's rapid metabolism and elimination. When six man and woman took an oral dose of 25 mg of trans resveratrol only traces of the unchanged resveratrol were detected in the plasma. Plasma concentrations of resveratrol and metabolites peaked around 60 minutes later at concentrations around 2 micromoles/liter.

The invention provides the addition of aspirin to the resveratrol which is believed to prolong and maintain the Cox1 inhibition in addition to all the other desired effects such as lowering the hyperlipidemia, maintaining good endothelial functions preserving muscle dilatation and anti hypertrophic stimulus as well as anti platelet aggregation and good protection of the stomach.

EXAMPLE 1

A pilot study of the method of invention was conducted on a woman, aged 68 years old that has been suffering from hyperlipidemia and ischemic heart disease. She underwent angioplasty and non medicated stent was introduced. The patient was then treated according to the method of the invention wherein aspirin (325 mg) and resveatrol (160 mg) were simultaneously given as the only daily treatment, for a period of six months.

History revealed no chest pains, no gingival bleeding, no black stool, no skin hematomas, no eye sight disturbances or any neurological symptoms. Stress ECG revealed no new ischemic changes. Cholesterol LDL went down from 300 to 150 and HDL went up and thrombaxane levels were reflecting optimal anti platelet aggregation.

In conclusion there appeared to be an excellent drop of LDL levels as well as good values of HDL. Thus, the combined therapy of aspirin and resveratrol was found to achieve optimal platelet aggregation inhibition and did not expose the patient to a risk of over anticoagulation. On top of it very good results were reached as far as the hyperlipidemia is concerned most probably due to the action of the resveratrol.

It is understood that this case is described for illustrative purpose and has no statistical significance nor can it teach us anything but it might only be the first patient treated with this new combination of drugs.

The foregoing description of various and preferred embodiments of the present invention has been provided for purposes of illustration only, and it is understood that numerous modifications, variations and alterations may be made without departing from the scope and spirit of the invention as set forth in the following claims.

What is claimed is:

1. A method for delivering a composition for use in treatment and prevention of vascular disease comprising the steps of:
providing a vehicle for delivery of the composition to an individual, wherein the composition contains a combination of aspirin and resveratrol;
administering said vehicle to the mouth of the individual, wherein said vehicle melts at the body temperature of the individual such that the aspirin and resveratrol is absorbed thru the oral mucosa and delivered directly into the inidvidual's bloodstream.

2. The method according to claim 1, wherein said vehicle is a medicinal melting capsule comprised of a material that has the same melting point as the human body temperature.

3. The method according to claim 1, wherein said vehicle is a solution of the combination of aspirin and resveratrol.

4. The method according to claim 1, wherein said vehicle is a tablet of the combination of aspirin and resveratrol.

5. The method according to claim 1, wherein said vehicle is a powder including the combination of aspirin and resveratrol.

6. The method according to claim 1, wherein said vehicle includes aspirin in the range of 81 to 325 mg.

7. The method according to claim 1, wherein said vehicle includes resveratrol in the range of 10 mg to 300 mg.

8. A melting capsule comprised of a material that has the same melting point as the human body temperature and includes aspirin in the range of 81 to 325 mg and resveratrol in the range of 10 to 300 mg.

9. The melting capsule of claim 8, further containing an outer layer to prevent the capsule from melting before use.

10. The melting capsule of claim 8, further containing an inner layer to prevent the capsule from melting before use.
